(19) United States
(12) Patent Application Publication
(10) Pub. No.: US 2008/0226029 A1
(43) Pub. Date: Sep. 18, 2008

(54) Medical device including scanned beam unit for imaging and therapy

(76) Inventors: Michael P. Weir, Blanchester, OH (US); Robert J. Dunki-Jacobs, Mason, OH (US); Neeraj P. Teotia, Cincinnati, OH (US); Paul G. Ritchie, Loveland, OH (US); Jere J. Brophy, Loveland, OH (US); Michael S. Cropper, Edgewood, KY (US); Thomas W. Huitema, Cincinnati, OH (US); Gary L. Long, Cincinnati, OH (US); Robert M. Trusty, Cincinnati, OH (US)

Correspondence Address: Christopher W. Elswick;Thompson Hine, LLP, 10 W. Second Street, 2000 Courthouse Plaza NE, Dayton, OH, 45402-1758, US

(21) Appl. No.: 11/716,806

(22) Filed: Mar. 12, 2007

(51) Int. Cl. A61N 5/10

(52) U.S. Cl. 378/65

(57) ABSTRACT

A medical device includes a radiation source assembly having at least two radiation sources, where one or more of the radiation sources is adapted to generate an imaging beam for use in visualization of a scene and one or more of the radiation sources is adapted to generate a therapeutic beam for treatment of a medical condition. An optical fiber for directing radiation energy from the radiation source assembly toward a distal end of the medical device in the form of a beam. A reflector that receives the beam from the optical fiber, the reflector configured to direct the beam onto a field-of-view. A receiving system including a detector arranged and configured to receive radiation from the field-of-view to generate a viewable image. The imaging beam and the therapeutic beam are directed to follow a common path from the at least two radiation sources to the reflector.

### TECHNICAL FIELD

The present application relates generally to medical devices and in particular to a medical device including a scanned beam unit configured for imaging and therapy.

### BACKGROUND

Various imaging devices have been used in medical procedures to allow a doctor to view a site within a patient. One such device described in U.S. Patent Publication No. 2005/0020926 is a scanned beam imaging system that utilizes a plurality of radiation sources, the outputs of which are sent to a distal tip via one or more optical fibers. The radiation is scanned across a field-of-view (FOV). The radiation reflected, scattered, refracted or otherwise perturbed within the FOV is gathered and converted into separate electrical signals that can be combined either electronically or through software and used to generate a viewable image.

### SUMMARY

In one aspect, a medical device includes a radiation source assembly having at least two radiation sources, where one or more of the radiation sources is adapted to generate an imaging beam for use in visualization of a scene and one or more of the radiation sources is adapted to generate a therapeutic beam for treatment of a medical condition. An optical fiber for directing radiation energy from the radiation source assembly toward a distal end of the medical device in the form of a beam. A reflector that receives the beam from the optical fiber, the reflector configured to direct the beam onto a field-of-view. A receiving system including a detector arranged and configured to receive radiation from the field-of-view to generate a viewable image. The imaging beam and the therapeutic beam are directed to follow a common path from the at least two radiation sources to the reflector.

In another aspect, a method of providing medical treatment is provided. The method includes outputting an imaging beam using a first radiation source and outputting a therapeutic beam using a second radiation source. The imaging beam is directed onto the field-of-view for generating a viewable image thereof using a reflector. The therapeutic beam is directed onto at least a portion of the field-of view based on specification of a target region.

In another aspect, a medical device includes a radiation source assembly configured to output an imaging beam and a therapeutic beam. An optical fiber is provided for directing at least one of the imaging beam and therapeutic beam toward a distal end of the medical device. A reflector receives at least one of the imaging beam and the therapeutic beam from the optical fiber. The reflector is configured to direct the at least one of the imaging beam and the therapeutic beam onto a field-of-view. A receiving system includes a detector configured to receive radiation from the field-of-view to generate a viewable image. A user input device allows for selection of a treatment region within the field of view.

The details of one or more embodiments of the invention are set forth in the accompanying drawings and the description below. Other features, objects, and advantages of the invention will be apparent from the description and the drawings, and from the claims.

### BRIEF DESCRIPTION OF THE FIGURES

FIG. 1 is a diagrammatic illustration of an embodiment of a medical device system including scanner assembly;

FIG. 2 is a diagrammatic illustration of an embodiment of a radiation source including multiple emitters for generating imaging, therapeutic and aiming beams;

FIG. 3 is a diagrammatic illustration of radiation paths in a system including a scanner assembly;

FIG. 4 is a diagrammatic illustration of an embodiment of a detector assembly;

FIG. 5 is a diagrammatic illustration of an embodiment of a controller for a medical device including a scanner assembly;

FIG. 6 is a perspective view of an embodiment of a scanner assembly for use with the medical device of FIG. 1;

FIG. 7 is a side, section view of the scanner assembly along lines 7-7 of FIG. 6;

FIG. 8 is a diagrammatic illustration of an embodiment of a radiation collector suitable for use with the medical device of FIG. 1;

FIG. 9 is a diagrammatic illustration of an endoscopic configuration of a medical device including a scanner assembly;

FIGS. 10-14 represent a variety of exemplary images and treatment regions;

FIG. 15 is a diagrammatic illustration of an embodiment of a user interface;

FIG. 16 is an illustration of a bisinusoidal scan pattern and a rectangular coordinate pattern plotted together;

FIG. 17 is a diagrammatic illustration of the user interactions with the medical device;

FIGS. 18 and 19 represents a conversion from Lissajous space to Cartesian space; and

FIG. 20 represents an exemplary sequence of conceptual timelines of various events during synchronized ON/OFF therapy.

### DETAILED DESCRIPTION

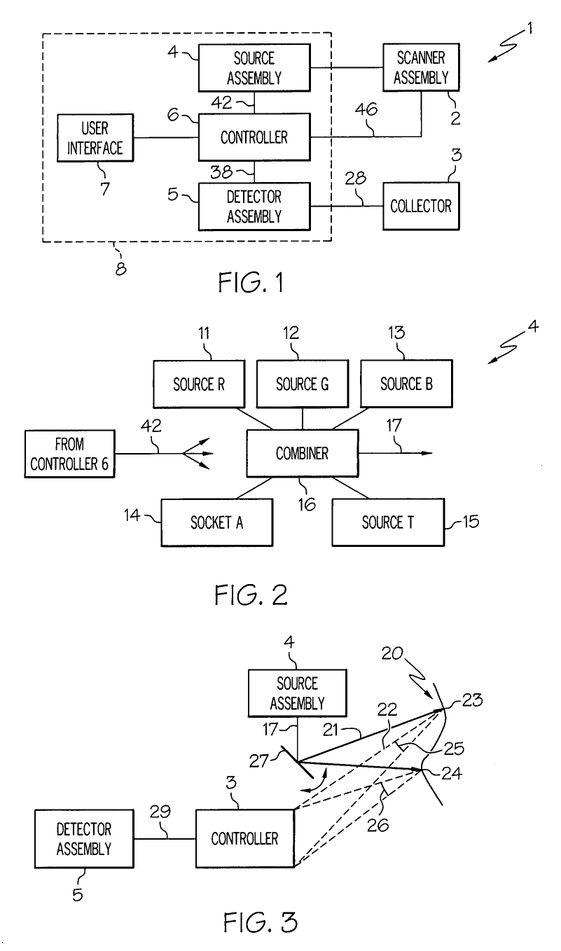

Referring to FIG. 1, an embodiment of a medical device 1 includes a scanner assembly 2, a collector 3, a radiation source assembly 4, a detector assembly 5, a controller 6 and a user interface 7. The radiation source assembly 4, detector assembly 5, controller 6 and user interface 7 make up functional element 8 that is known herein as a “console.” The radiation source assembly 4, as selected by the user via the user interface 7, and acting through the controller 6, generates at least two wavelengths of radiation (e.g., in the visible wavelength range and/or otherwise). This radiation is conveyed in a beam to the scanner assembly 2, which causes the beam to be swept across a tissue surface. The extent of this swept area is generally known as the “field of view” (FOV). Radiation reflected from the scene within the FOV may be intercepted by the collector 3 and passed to the detector assembly 5. The detector assembly converts the received radiation to electrical signals that are then configured by the controller to form an image on a display device in the user interface 7.

FIG. 2 is a block diagram of one implementation of the source assembly 4. Source assembly 4 includes multiple sources, each capable of generating radiation at a selected wavelength. Five sources are shown here, numbered 11 thru 15. The outputs of the radiation sources 11-15 may, in some embodiments, be brought together in combiner element 16 to yield an output beam 17. Combiner 16 may also include beam-shaping optics such as one or more collimating lenses and/or apertures. The sources may be of various types such as, for instance, light emitting diodes (LEDs), lasers, thermal sources, arc sources, fluorescent sources, gas discharge sources, or others. Signals 42 may be provided by controller 6 (FIG. 1) to one or more of the sources and optionally the combiner 16. Signals 42 may optionally control wavelength, power, modulation or other beam properties. The wavelength of radiation, for example, may be selected for imaging, therapy, or aiming. As used herein, an “imaging beam” refers to radiation selected for use in creating an image of a surface or region, a “therapeutic beam” refers to radiation selected to provide treatment of a condition such as diseased or damaged tissue, and an “aiming beam” refers to radiation selected to accentuate a portion of the FOV. In this example, sources 11, 12 and 13 emit red, green and blue radiation; source 14 emits an aiming beam at a wavelength selected to yield a distinct contrast to the typical target material; and source 15 emits a therapeutic beam at a wavelength that is highly absorbed and moreover can be efficiently generated at high power to treat diseased or damaged tissue. In some embodiments, the aiming beam may be provided by source separate from the therapeutic beam source 15. As an alternative, an aiming beam may be provided by source 15 as a reduced power therapeutic beam. In some embodiments, the aiming beam could be a virtual beam (i.e., a region in which one or more of the imaging sources is caused to increase (or decrease) significantly to create a bright (or dark) region in the displayed image.

In some embodiments, a source (not shown) provides a diagnostic beam. A “diagnostic beam” as used herein refers to radiation selected for analysis or detection of a disease or other medical condition including, for example, to visualize the presence of (or to activate) a diagnostic marker. The diagnostic marker could be naturally occurring (e.g., auto or self fluorescence) or introduced as part of the diagnostic procedure (e.g., fluorescent dyes).

Use of an aiming beam may be preferred in some circumstances. As will be seen later, while the treatment beam may follow the same path as the imaging beam, it is not constrained to follow the same timing. An aiming beam, managed in the same way as the therapeutic beam though at lower power and in a visible wavelength, may help ensure that the treatment is applied where the user intends. Furthermore, it may be a requirement of certain industry or regulatory standards such as AAMI or IEC that where higher power lasers are employed, an aiming beam be provided.

It should be noted that while five sources are illustrated, there may be more or fewer emitters depending, for example, on the end use. In some embodiments, sources may be combined or capable of providing various types of energy. In some cases, filters may be used to filter the radiation. In some embodiments, sources 11, 12 and 13 comprise three lasers; a red diode laser, a green diode-pumped solid state (DPSS) laser, and a blue DPSS laser at approximately 635 nm, 532 nm, and 473 nm, respectively. While laser diodes may be directly modulated, DPSS lasers generally require external modulation such as an acousto-optic modulator (AOM) for instance. In the case where an external modulator is used, it is considered part of the radiation source assembly and not shown separately.

FIG. 3 illustrates the operation of a device 1 incorporating a scanner assembly 2. Reflector 27, part of the scanner assembly 2 to be described in more detail later, receives a beam of radiation 17 from source assembly 4 and directs the beam onto the surface 20, for example, for one or more of imaging, therapy, or aiming purposes. At one point in time, the beam deflected by the reflector 27 is in direction shown as 21, and impinges upon the surface to illuminate a point 23. Reflector 27 oscillates in at least one axis (two axes in some embodiments), as indicated by the nearby arrowed arc, so that at some other point in time the deflected beam is in the direction indicated as 22 where, it illuminates point 24. Radiation is, in general, reflected, absorbed, scattered, refracted or otherwise affected by the properties of the surface. Radiation may leave the surface in many directions. The collector 3, however, may only capture that fraction of radiation which falls into the area subtended by its aperture. Regions 25 and 26 show the reflected radiation that is captured by the collector 3 when the beam is illuminating points 23 and 24 respectively. Directions 21 and 22 are not intended to represent any special part of the scan as the beam may be scanned using reflector 27 beyond them, and scans all points between them as well. Furthermore, a simplified two-dimensional view is represented by FIG. 3, and in general reflector 27 and collector 3 are adapted to illuminate and capture from surfaces occupying space in three dimensions.

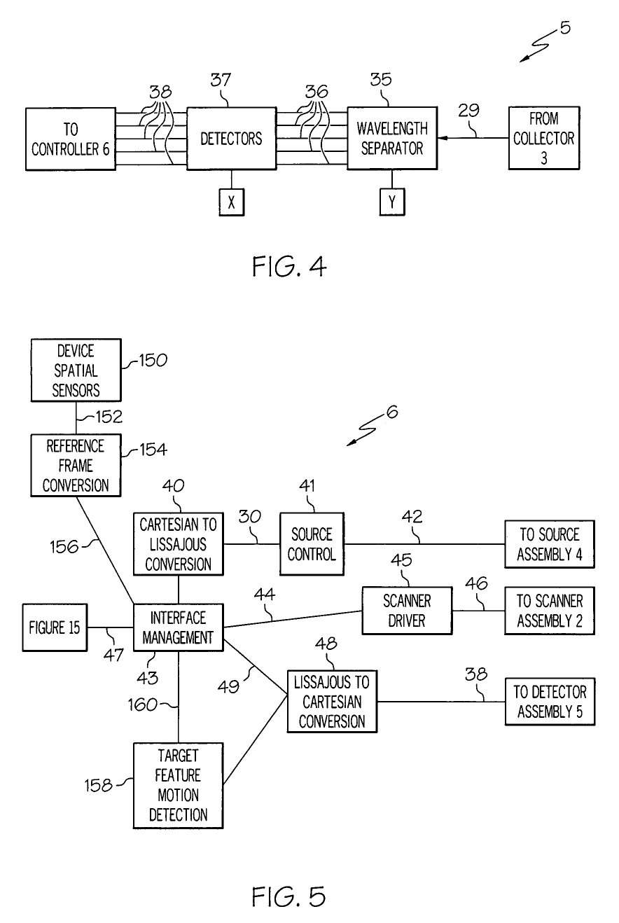

FIG. 4 is a block diagram of the exemplary detector assembly 5. Radiation 29 that is intercepted by the collector 3 is passed to the detector assembly 5. This radiation includes energy at several wavelengths, corresponding to those emitted by the source assembly 4, and possibly also including other wavelengths as may result from nonlinear processes (such as fluorescence). In some embodiments, wavelength separator 35 separates the incoming radiation 29 into pathways 36. Such separation may be performed by filters, gratings, or other devices. In an alternate configuration, wavelength separation may be incorporated in the collector 3, and separated wavelengths brought to the detectors 37, each in its own fiber or fiber bundle. Each separated wavelength of radiation is then sent to detectors 37 in the detector assembly 5. Such detectors may be physically separate, or parts of a common detector such as a CCD or CMOS device. Multiple detectors 37 may be incorporated for each wavelength. The detectors output electrical signals 38 corresponding to the power, amplitude, or other characteristic of each wavelength of radiation detected. The signals can be used by a controller 6 (FIG. 5) to generate a digital image, e.g., for processing, decoding, archiving, printing, display, etc.

In some embodiments, X represents an input to the detectors 37 capable of modifying the transfer function from radiation to electric signals. Exemplary modifications may include adjustment of gain or offset or both. Y may represent an input to the wavelength separator 35 capable of modifying the transfer function therethrough. The modifying elements X and Y may be disposed to operate on the input to the respective detectors 37 and wavelength separator 35, acting on all or a subset of wavelengths received, at the outputs of the respective detectors 37 and wavelength separator 35 or at both inputs and outputs.

FIG. 5 is a block diagram of the exemplary controller 6. An interface management component 43, among other tasks, accepts operating mode commands from the user, illustrated as part of path 47. Such commands may include imaging and treatment modes, FOV and/or aspect ratio of the image, image storage, etc. Specifications related to the FOV and aspect ratio result in parameters sent via path 44 to a scanner driver 45, which generates requisite drive signals 46 to the reflector 27. The user may also specify treatment parameters, such as the location, shape and size of a region to be treated, the wavelength to be used, and duration of exposure. These result in parameters being sent to a coordinate converter 40, which converts the specifications into selection and modulation commands 30 to a source control and modulation block 41. This source control and modulation block 41 drives the source assembly 4 to provide the requisite radiation outputs 17. Signals 38 from the detector assembly 5 are converted from their scan coordinate system to a Cartesian form 49 at block 48 for display and sent to the interface management block 43 for user viewing. Details of this conversion procedure are described later.

In some embodiments, motion sensing is incorporated within the system. For example, element 150 may include a number of sensors attached or connected to the scanner assembly 2. The sensors may sense location, orientation or both. The sensors may be, for example, accelerometers, magnetometers, rate gyros, electromagnetic position sensors, etc. Element 152 represents the location and orientation signals generated by the sensors and element 154 represents a mathematic operation capable of converting the signals 152 into a stationary reference frame. Element 156 represents output of element 154 which is used to modify the relationship of a displayed image to the scanned data 49 to compensate for sensed movement.

Element 158 operates on the scanned data 49 to detect the relative movement and provides signals 160 indicating magnitude and direction of the movement. This image tracking functionality may provide reliable treatment of the body which might be moving due to, for example, respiration, circulation or other biological activity.

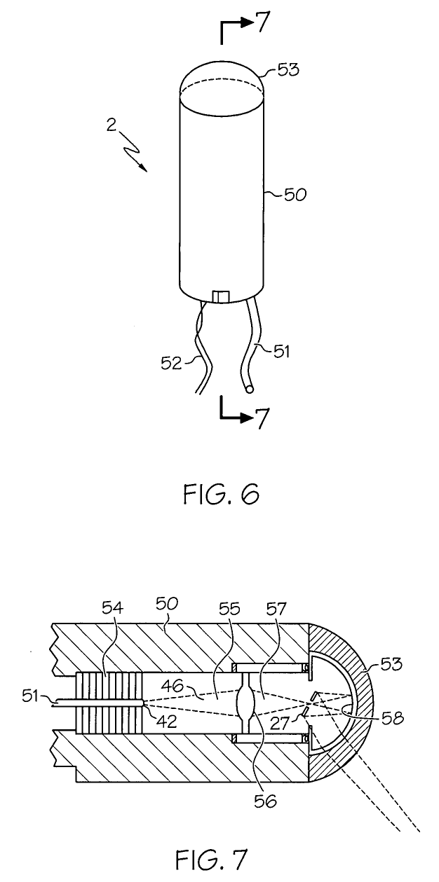

FIG. 6 is an external view of one embodiment of the scanner assembly 2. Scanner assembly 2 includes a housing 50 that encloses the reflector 27 and other components. A source fiber 51 is used to deliver energy from the source assembly 4 to the scanner assembly 2. Source fiber 51 may be a single mode optical fiber. In some embodiments, one or more fibers may be used to deliver imaging beams and one or more other fibers may be used to deliver a therapeutic beam (e.g., therapeutic beams having longer wavelengths, e.g., greater than 1700 nm and/or higher power). In certain embodiments, a different type of fiber, such as a holey fiber, may be used to transmit energy from the source assembly 4. In some embodiments, the same optical fiber 51 is used to deliver both the imaging beams and the therapeutic beams to the reflector, the optical fiber defining a common path for both types of beams.

Electrical wires 52 convey drive signals for the reflector 27 and other signals (position feedback, temperature, etc.) to and from the scanner driver 45 (FIG. 5). Wires 52 may also provide control and feedback connections for controlling focus characteristics of the beam shaping optic 56. The distal end of the scanner assembly 2 is fitted with an optical element 53 which allows the scanned beam to pass out and illuminate the scene. This element 53 is generally referred to and illustrated as a dome; however, its curvature, contour, and surface treatments may depend on the application and optical properties required. In some embodiments, dome 53 provides a hermetic seal with the housing 50 to protect the internal elements from the environment.

FIG. 7 shows internal components of an embodiment of the scanner assembly 2. Source fiber 51 is affixed to the housing 50 using a ferrule 54. The end of the source fiber 51 may be polished to create a beam 55 of known divergence. The beam 55 is shaped by a beam shaping optic or lens 56 to create a beam shape appropriate for transmission through the system. After shaping, shaped beam 57 is fed through an aperture in the center of reflector 27, then reflected off a first reflecting surface 58. First reflecting surface 58 may have a beam shaping function. Beam 57 is then directed onto reflector 27 and then out of the scanner assembly 2, the details of which (in the case of an imaging beam) are described in U.S. patent application Ser. No. 10/873,540, entitled SCANNING ENDOSCOPE, the details of which are hereby incorporated by reference as if fully set forth herein. Any suitable materials can be used to form the reflector 27. In some embodiments, the reflective surface of the reflector 27 may be formed of gold or other suitable material for directing each of the beams including relative high energy therapeutic radiation. In other embodiments, a multilayer dielectric configuration may be used in forming reflector 27.

FIG. 8 shows an embodiment of the collector 3, which in this case is configured to be installed coaxially with the scanner assembly 2. Radiation reflected from a scene impinges on the face 60 of the collector 3, which constitutes the receiving aperture. Face 60 is actually made up of the polished ends of a large number of small diameter, multimode collecting fibers 63 which conduct the radiation to the detector assembly 5. Scanner assembly 2 is inserted into a central void 61. The collector 3 is enclosed by a housing 62. The fiber ends making up face 60 may be formed in a plane, or into other geometries to control the pattern of receiving sensitivity. They may be coated with diffusing or other materials to improve their angle of acceptance, to provide wavelength conversion, or wavelength selectivity. In some embodiments, the detector assembly 5 may be configured to form the receiving aperture and mounted in position to receive the reflected radiation directly, without the need for a separate collector 3.

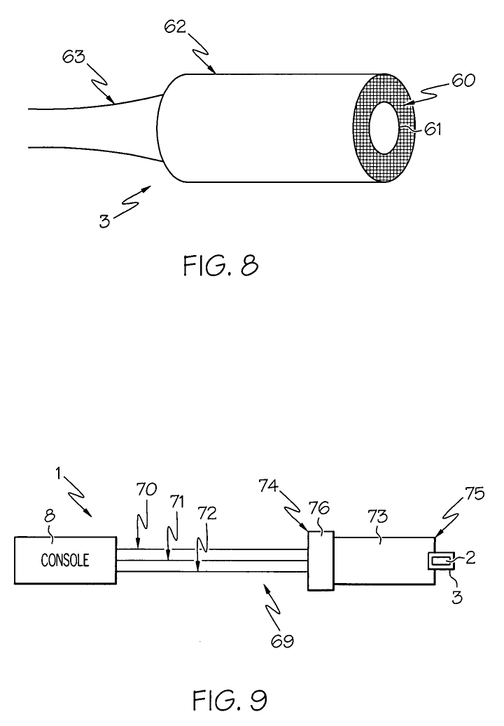

FIG. 9 shows diagrammatically various elements previously described as incorporated into an exemplary endoscope 69 for medical use. Endoscope 69 generally includes an elongate, rigid or flexible shaft 73 having a distal end 74 and a proximal end 75 opposite the distal end. There is typically a handle 76 which includes a number of controls, often both mechanical and electrical. The endoscope 69 is connected to console 8 by source fibers 70, collection fibers 71, and electrical wiring 72. As used herein, an endoscope refers to an instrument for use in examining, diagnosing and/or treating tissue comprising a patient's body, either percutaneously or through a natural orifice or lumen. As used herein, the term “proximal” refers to a location on the medical device nearer to a user, and the term “distal” refers to a location that is nearer the patient. Typically, the console 8 of the medical device 1 is located outside a patient's body and the distal end of the medical device is insertable into the patient's body. However, other configurations are possible. Furthermore, while an endoscope is referred to, any suitable type of medical device may be employed such as gastroscopes, enteroscopes, sigmoidoscopes, colonoscopes, laryngoscopes, rhinolaryoscopes, bronchoscopes, duodenoscopes, choledochoscopes, nephroscopes, cystoscopes, hysteroscopes, laparoscopes, arthroscopes, etc.

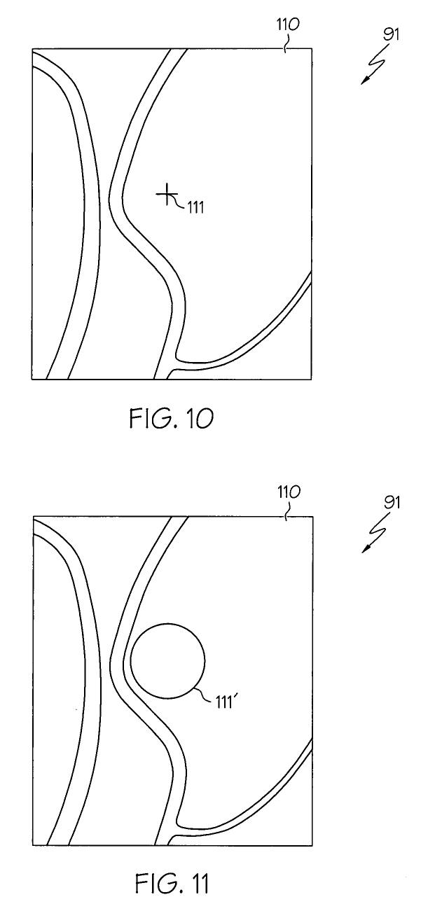

FIGS. 10-14 represent diagrammatically various exemplary images and treatment regions and FIG. 15 is a diagrammatic illustration of an embodiment of a user interface for use in selecting a desired treatment region, where applicable. Referring first to FIG. 10, one exemplary mode of operation of the system in performing therapy is illustrated. An image 110 of the scene is displayed on geometry display device 91. Controller 6 generates a cursor 111 illustrating where the treatment beam will be emitted. The aiming beam may be enabled to confirm the point of treatment before enabling the treatment beam. In this mode, the treatment beam occupies a fixed position in the display space, and the operator manipulates the scope so as to bring the tissue to be treated into alignment with that beam. The treatment zone is represented as being small, such as might be the case when an incision or cauterization is planned along a line.

FIG. 11 represents a similar mode of operation to that described in FIG. 10, with the difference that the user has employed a geometry input device 93 (FIG. 15) to specify a region 111′ over which treatment is to take place, represented here as a circle.

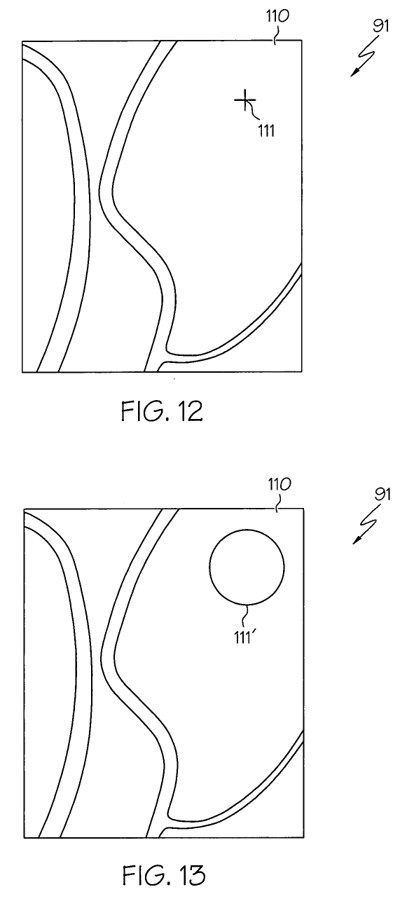

FIG. 12 represents a similar mode of operation to that described in FIG. 10, except that the cursor 111 can be positioned at a location selected by the user. In this embodiment, the device 1 (e.g., endoscope 69) is positioned such that the desired treatment point and important other details of the scene are visible in the image 110. The user can then position the cursor 111 at the location of the desired treatment point (e.g., by touching the geometry display device 91 at the desired location or by using geometry input device 93; FIG. 15).

FIG. 13 represents a similar mode of operation to that described in FIG. 12, except that the user has employed the geometry input device 93 (FIG. 15) to specify a region 111′ over which treatment is to take place, represented here as a circle.

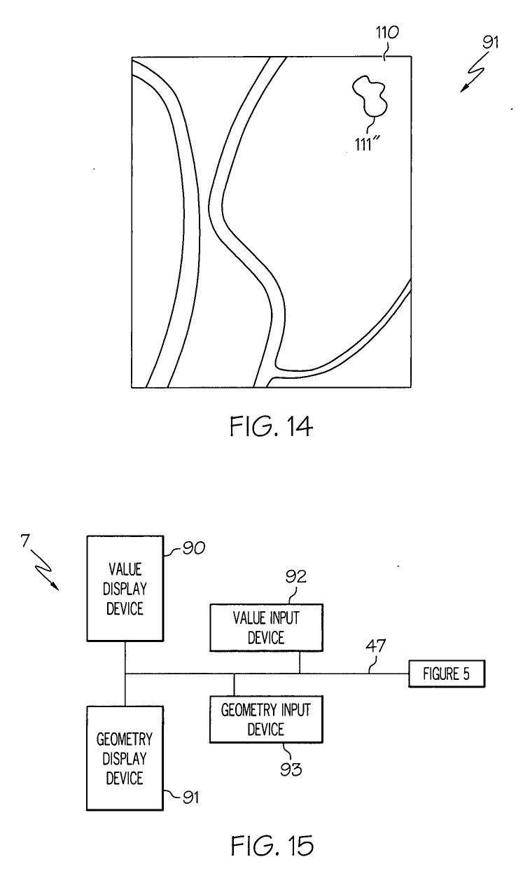

FIG. 14 represents a similar mode of operation to that described in FIG. 13, except that the user has specified an irregular region 111″ of treatment. Such regions may be defined by specification of a number of points, using geometry input device 93 (FIG. 15), between which the system constructs lines or curves defining the treatment boundary, or by stretching and modifying a small number of predefined geometric shapes. In such a mode, the aiming beam is particularly useful in confirming that the treatment region will be where the user intended.

It should be noted that while a single treatment zone or region is shown specified in FIGS. 10-14, in some embodiments, multiple treatment zones may be specified simultaneously.

FIG. 15 shows, in general terms, the user interface 7. The expression “value” in this figure refers to quantities which can be expressed as simple numbers, text strings or scalar quantities. The expression “geometry” refers to quantities that have a multidimensional or multiparameter nature, such as an image, a vector, an area, or a contour. Commands 47 from the interface management block 43 (FIG. 5) are displayed for user viewing on the value or geometry display devices 90 or 91 respectively. Referring also to FIG. 5, interface management 43 may be software (and possibly dedicated hardware) to manage and control the data to and from the devices in FIG. 15. Interface management 43 includes control logic to manage the setting of treatment point 111 and, once a treatment is determined and requested, to control the creation of the control sequences to cause treatment through 40 and 41. Examples of value quantities that might be displayed include operating mode, power output, equipment status, field of view, available image storage space, date, and time. Geometry display quantities include the image of the scene being viewed, treatment regions, boundaries, or paths. Input values include operating mode selection, names of stored image files, and image (color balance, brightness, contrast, enhancement) adjustments. Geometry input quantities include a region or pathway to be treated by a therapeutic beam, or zones in which special image processing is to be performed.

All these functions may be provided in a single multifunction device, such as a touch screen panel, which can accept user input as well as display data of geometric and value types. It may be preferable, however, to provide specialized devices which provide a more ergonomic or haptic match to the operating tasks. For example, a text display might be utilized for value display 90, reserving a larger and more expensive graphical display for the geometry display 91 to avoid cluttering images with interfering data. Similarly, while simple pushbuttons or keyboards (virtual or real) may serve to enter both values and geometry quantities, they may be ill suited to the latter. A joystick, trackball or multi-axis device such as used on the Da Vinci surgical robot, available from Intuitive Surgical, Inc. may be used for specifying geometry inputs.

In addition to marking a region and then providing a signal to the scanner assembly 2 to apply the therapeutic beam to that region, a more interactive and immediate treatment mode may be provided, where the geometric input device is used to enable real-time, live application of treatment radiation, typically in a small spot beam such as would be familiar to users of electrocautery and laser cutting devices. Such a mode may be useful in a variety of surgical procedures such as prostate surgery which can be performed under direct visualization without additional cystoscopes, bladder surgery where bladder tumors or bladder cancer can be imaged and thermally necrosed, removal of varicose veins where the endoscope 69 can be inserted into the saphenous vein and used to treat the inside of the vein under direct visualization, destruction of kidney stones where the stone can be directly visualized and then thermally destroyed using a single device, etc.

In one embodiment, a treatment region may be automatically recognized, for example, using the presence of fluorescence or other marker. A signal may then be provided to the scanner assembly to apply the therapeutic beam to that automatically selected treatment region. A disease or tissue specific agent bound to a fluorescent material may be placed into the patient via, for example, the circulatory system that gathers at the target diseased or tissue area. The system can automatically identify a region to be treated by observing, for example, a spectral signature of the fluorescent material. The user may then confirm the treatment region and then authorizing treatment to proceed (possibly specifying a treatment dose).

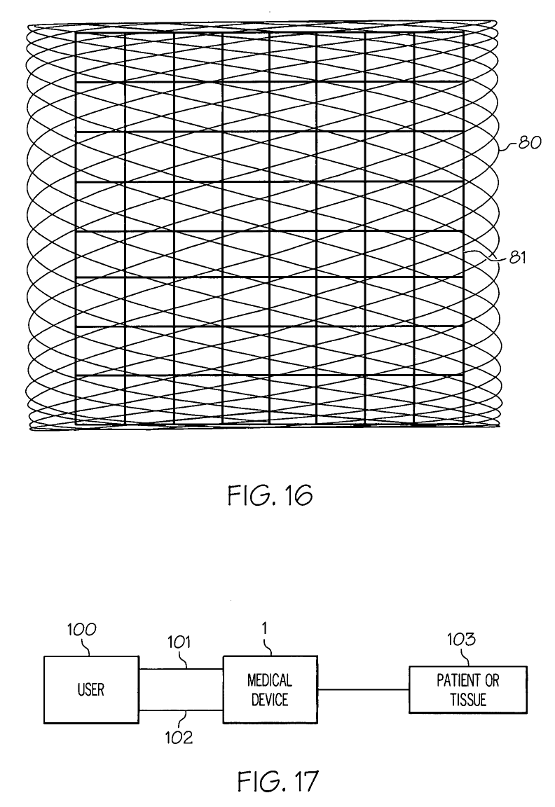

Referring now to FIG. 16, as mentioned above, the reflector 27 scans the beam of radiation in a pattern. FIG. 16 shows an idealized bi-resonant or bi-sinusoidal scan pattern. High-speed MEMS reflectors and other resonant deflectors as described herein are configured and driven to execute sinusoidal angular deflections in two orthogonal axes, yielding the classical Lissajous pattern. Most current display devices (such as those diagrammatically represented by FIGS. 10-14) are configured to address display data in a Cartesian form, for example as row and column, or a particular pixel along a nearly-horizontal scan line. The bi-resonant or Lissajous scan path 80 is shown overlaid with the Cartesian or rectilinear grid 81. In the illustrated instance, the intersections between the vertical and horizontal lines of the Cartesian grid 80 represent display pixel positions while the Lissajous trace 81 represents the actual path taken by the scanned spot. As the actual scan path does not align perfectly with all the rectilinear pixel positions, these image values may be determined through interpolation. In some embodiments, registration of the Lissajous trace 80 to the Cartesian grid 81 is based on a marker that links a reference point in the scan to a point in the rectilinear matrix.

FIG. 17 shows the interaction between a user 100 and the system. User 100 defines a treatment zone, border, or path by means of specification 101 while viewing the image 102. Such specification includes the identification of places in the image, and thus on the target tissue 103, and selection of parameters such as the treatment beam wavelength, power, and duration of exposure. In FIG. 17, specification 101 and image 102 are represented as data quantities passed between system elements; in other figures, they may be represented as planar figures.

The user may define a treatment zone, border and/or path to perform and one or more of a variety of medical procedures. A general discussion of various laser treatment modalities using source 15 follows. This discussion is not meant to be exhaustive and should not be construed as limiting. Generally, laser therapy can be categorized into four areas: (i) Photodynamic Therapy (PDT), (ii) dermal treatment, (iii) thermal ablation and (iv) opto-thermal shock waves. A discussion of complexities involved in designating a treatment zone and delivering the desired treatment to that treatment zone using the scanner assembly 2 follows.

In PDT, a chemical (e.g., porfimer sodium) that preferentially collects at a target organ or tissue type is introduced into a patient, typically intravenously. The chemistry may be such that it is relatively inert until it is activated photonically. A therapeutic laser beam of the appropriate wavelength and power (typically visible wavelengths such as between about 400 nm and 700 nm and moderate power such as between about 1 mW and 100 mW) is caused to illuminate the target tissue, which activates the chemical and treats the tissue, typically through oxidative destruction of tumors located in the tissue.

In dermal treatments, a wavelength is typically selected to be preferentially absorbed by the targeted tissue or material to be treated, and energy density is selected to ablate the target material without unduly destroying adjacent tissue. For example, in tattoo removal, different color dyes absorb specific laser wavelengths and the laser power is chosen to vaporize the dye encapsulated in the tissue, causing the dye color intensity to diminish. Tattoo removal using a scanned beam imager is described in U.S. Ser. No. 11/615,140, entitled APPARATUS AND METHOD FOR MEDICALLY TREATING A TATTOO, filed Dec. 22, 2006, the details of which are hereby incorporated by reference as if fully set forth herein.

In thermal ablation, specific tissue is targeted for volumetric necrosis. Tissue necrosis is accomplished by subjecting tissue cells to a particular temperature for a particular period of time. Thermal ablation can be sub-categorized into several regimes such as coagulation and vaporization. During heating, the tissue is heated to temperatures generally less than about 41° C. with no lasting effect results. During coagulation, the tissue is heated to between about 41° C. and 100° C., and cell death occurs based on the amount of time the tissue is subjected to the temperature. Generally in coagulation, a wavelength may be chosen to maximize tissue penetration depth to evenly heat a volume, for example, in the near infrared between about 700 nm and 1050 nm and at lower power levels, such as between about 1 W and 50 W. In vaporization, a wavelength is typically chosen to be absorbed at the surface of the targeted tissue, and the low volume of cells at the tissue surface experience rapid temperature rise above 100° C., and the tissue is immediately denatured and vaporized. For vaporization, power levels can vary greatly based on the energy density delivered to the tissue, but are typically between about 1 W and 50 W. In opto-thermal shock, a laser is chosen with a fast pulse time such that very high instantaneous energies are used to create cavitation bubbles that collapse quickly and send a mechanical shock wave through targeted tissue. This type of treatment is typically used in laser lithotripsy to break up stones or calcification sites in the patient. Q-switched Nd:YAG (e.g., 1060 nm) or Alexandrite (e.g., 380 nm, 760 nm, 1440 nm), erbium:YAG (or Ho:YAG, e.g., 2112 nm) lasers may be suitable for opto-thermal shock treatment with sub-microsecond pulse times (e.g., 8 ns). Flash-lamp-pulsed dye lasers may also be suitable with longer pulse times on the order of 1-250 us. In some cases, CW lasers may be used in lithotripsy to heat a stone directly to cause stress-induced breakage.

Therapeutic beam modulation may be employed to deliver the desired amount of therapeutic radiation as the reflector 27 moves along its scan path. Generally, a beam which has been deflected by a mechanically-resonant reflector moves through space at a varying velocity. When this beam impinges upon a target, the time spent in any one area may differ across the FOV. Additionally, the size of the spot (or footprint) on the target may vary with the target's distance and inclination to the beam, which can cause the flux to vary. Various therapeutic beam modulation schedules due to variable velocity and beam footprint size are discussed in U.S. Ser. No. ______, entitled POWER MODULATION OF A SCANNING BEAM FOR IMAGING THERAPY AND/OR DIAGNOSIS, filed on the same day as the instant application [attorney docket no. END5900USNP], the details of which are hereby incorporated by reference as if fully set forth herein.

As can be appreciated, complexities may arise when both imaging and delivering therapeutic radiation. While collecting image data, the illumination power may be on the order of milliwatts or tens of milliwatts, depending on the application requirements (working range, field of view, etc.). The treatment power, on the other hand may be in the range of watts or tens of watts. The treatment power may be delivered at wavelengths outside the visible range, or within the visible range, and may even be within the range of those wavelengths used for imaging. It will be apparent that even though the treatment wavelengths are selected for tissue effect, meaning they must be significantly absorbed into the tissue, the target may reflect significantly higher treatment energy than imaging energy.

All systems having inputs, but particularly receiving and detecting systems, may be characterized by their dynamic range. Various definitions are used depending on context and application, but all include the notion of a range of signal levels over which they operate correctly. Typically, if signals are below the lower limit of the range, they are not seen as distinguishable from noise. If the signals are above the upper limit of the range, then they will not be correctly recognized. In many detection systems, such as would be employed in a scanned beam system, the detection system may be “saturated” or “paralyzed” by signals above the upper limit, meaning that the detection system does not respond even to signals within its dynamic range, for some extended period of time. The detection system may recover to full functionality after some prolonged period of recovery. If signals are too high, the detection system may be permanently damaged. “Overload” is a term often applied to situations where the signal is beyond the upper limit of the dynamic range, and the overload may be so large as to damage the detection system.

When treatment wavelengths are well separated from those employed for imaging, high-pass, low-pass, and band-pass filters may be appropriately used to inhibit any damaging amount reflected treatment power making its way through the receiving elements to the imaging detectors. When the treatment wavelengths are near the imaging wavelengths, however, filters may be of less utility because of practical constraints on the accuracy, sharpness and stability of their transfer characteristics. Furthermore, even when the wavelengths are well separated, the amount of attenuation out of band is not infinite, and some treatment energy may leak into the imaging system. Finally, since the treatment and imaging beams are likely to be in close proximity, sharing deflection and other system components, the probability of scattering some treatment energy into the imaging system even before it impinges on the target is high.

Thus, it may be advantageous to design for some small amount of the treatment energy to leak into the imaging system, for it provides irrefutable confirmation of the region experiencing treatment. Nevertheless, it may be appropriate to employ further measures to inhibit excessive disruption of the receiving system. In many cases, it may be the detector elements which are most susceptible. A number of means for inhibiting disruption of the detection system may be employed (see FIG. 4). For example, the sensitivity of the detectors 37 may be reduced when the treatment energy is applied, for example by reducing or removing bias on avalanche photodiodes. The amount of energy input to the detectors 37 may be attenuated, for example by using electrooptic modulators. The input to the detectors 37 may be completely blocked, for example, by using MEMS devices such found in the Texas Instruments Digital Light Projector to deflect the energy away from the detectors.

In addition to protecting the detector 37 from overload, circuitry following the detector may also be configured to prevent generation or propagation of any large transient that accompanies the systems and processes just described. A suitable approach, for example, is to preserve and hold the signal present just before the onset of treatment, and return to following the input when the treatment ceases. This mechanism may be applied immediately after the detector 37 in the signal processing chain. Further modifications of the signal, for example, to show a pseudocolor or other indicator of treatment in progress, may be applied here as well, or later in the signal processing chain.

Some embodiments use a micro-electromechanical (MEMS) scanner reflector to direct the imaging, aiming and therapeutic beams onto the surface. MEMS scanner reflectors are described in, for example, U.S. Pat. No. 6,140,979, entitled SCANNED DISPLAY WITH PINCH, TIMING, AND DISTORTION CORRECTION; U.S. Pat. No. 6,245,590, entitled FREQUENCY TUNABLE RESONANT SCANNER AND METHOD OF MAKING; U.S. Pat. No. 6,285,489, entitled FREQUENCY TUNABLE RESONANT SCANNER WITH AUXILIARY ARMS; U.S. Pat. No. 6,331,909, entitled FREQUENCY TUNABLE RESONANT SCANNER; U.S. Pat. No. 6,362,912, entitled SCANNED IMAGING APPARATUS WITH SWITCHED FEEDS; U.S. Pat. No. 6,384,406, entitled ACTIVE TUNING OF A TORSIONAL RESONANT STRUCTURE; U.S. Pat. No. 6,433,907, entitled SCANNED DISPLAY WITH PLURALITY OF SCANNING ASSEMBLIES; U.S. Pat. No. 6,512,622, entitled ACTIVE TUNING OF A TORSIONAL RESONANT STRUCTURE; U.S. Pat. No. 6,515,278, entitled FREQUENCY TUNABLE RESONANT SCANNER AND METHOD OF MAKING; U.S. Pat. No. 6,515,781, entitled SCANNED IMAGING APPARATUS WITH SWITCHED FEEDS; U.S. Pat. No. 6,525,310, entitled FREQUENCY TUNABLE RESONANT SCANNER; and U.S. patent application Ser. No. 10/873,540, entitled SCANNING ENDOSCOPE; all of which are hereby incorporated by reference in their entirety as if fully set forth herein.

As shown in FIG. 17, user 100 defines a treatment zone, border, or path by means of specification 101 while viewing the image. Such specification may include the identification of places in the image, and thus on the target tissue. This action can be performed through tracing paths on the geometry display device 91, utilizing the geometry input device 93 as noted above. These paths generally follow the periphery of regions to be treated. The controller 38 can maintain and mark the selected path, and allow adjustment and editing of the path.

A further task in establishing the treatment domain is selection of parameters such as the treatment beam wavelength, power, and duration of exposure. In some embodiments, the operator utilizes the value input device 92 to complete these tasks.

The following discussion describes how specification of points in the display space, from which lines and then areas may be specified, may be mapped to the acquisition space. The discussion begins with mapping from scan coordinates to display coordinates and then from display coordinates (e.g., where a user has specified a treatment region) to scan coordinates (e.g., where the treatment radiation is to be applied).

Scan Coordinate to Display Coordinate Mapping

The scanner assembly 2 employs an oscillating reflector 27 with two orthogonal axis of rotation (labeled x and y) that operate in a resonant mode. The rate of oscillation is typically higher in one axis than the other. When properly excited, the oscillating reflector 27 causes a beam of light reflected from its surface to trace a Lissajous pattern. The coordinates of the beam are approximated by

x(t)=A sin(w_{f}t+φ_{f})

y(t)=B cos(wₛt+φₛ).

Based on the phase relationship of the slow and fast axis motion, the basic Lissajous pattern can precess. The number of slow axis cycles required to precess the pattern to an initial spatial point, is called the interleave factor.

The Lissajous pattern is spatially repeated after a set number of oscillations on the slow axis (interleave factor). Once a reference point on the complete set of Lissajous patterns is identified, one can view the constant sample time, digital data stream captured at each optical detector as a vector of constant length, the Scanned Data Vector (SDVᵢ). The number of samples in the vector (N) is equal to the interleave factor times the period of the slow axis oscillation divided by the sample interval (tₛ).

SDVᵢ(jΔt)=[s(i, j)]ⱼ₌₀^{N-1}

If there are multiple optical detectors sampled coincidently, then the scanner assembly data stream can be viewed as a matrix, the Scanned Data Matrix (SDM), that has a row count equal to the number of sampled detectors (M) and a column count equal to the number of samples in each SDV (N). In a system having three color plus fluorescence channels,

$$SDM = [ \begin{matrix}{SDV}_{R} \\ {SDV}_{G} \\ {SDV}_{B} \\ {SDV}_{F}\end{matrix} ] .$$

The pixel data matrix (PDM) is a two-dimensional matrix with row and column indices that represent the display space. In the above-described scanner assembly 2, for example, there may be 600 rows (Y) and 800 columns (X) and each point in the data set may be a triple representing red (R), green (G), and blue (B) display intensities.

$$PDM = [ \begin{matrix}( {r}_{0 , 0} , {g}_{0 , 0} , {b}_{0 , 0} ) & ⋯ & ( {r}_{0 , 799} , {g}_{0 , 799} , {b}_{0 , 799} ) \\ ⋮ & & ⋮ \\ ( {r}_{599 , 0} , {g}_{599 , 0} , {b}_{599 , 0} ) & & ( {r}_{799 , 599} , {g}_{799 , 599} , {b}_{799 , 599} )\end{matrix} ]$$

In order to conveniently describe matrix operations, it may be useful to define a view of the matrix, PDM, that is a vector of length XY called PDV. The transformation between the two is not a matrix operation, but rather a reordering where the rows of PDM are constructed of successive blocks of PDV. Note that it is essential that the same reordering be used when accessing the PDV and the transformation matrix, T to be described next.

One exemplary method for transforming between Lissajous and Cartesian spaces involves multiplication by a matrix T or its inverse. The process for constructing this matrix is given in a later section. Matrix T is an Nx XY matrix where N is the number of samples in the SDV, X is the number of horizontal pixels in the display space; and Y is the number of vertical pixels in the display space.

When converting from the Lissajous space SDM to the Cartesian space PDM, it may be helpful to take a close look at the physical situation from which the data derives. FIG. 23 provides the basis for the following discussion.

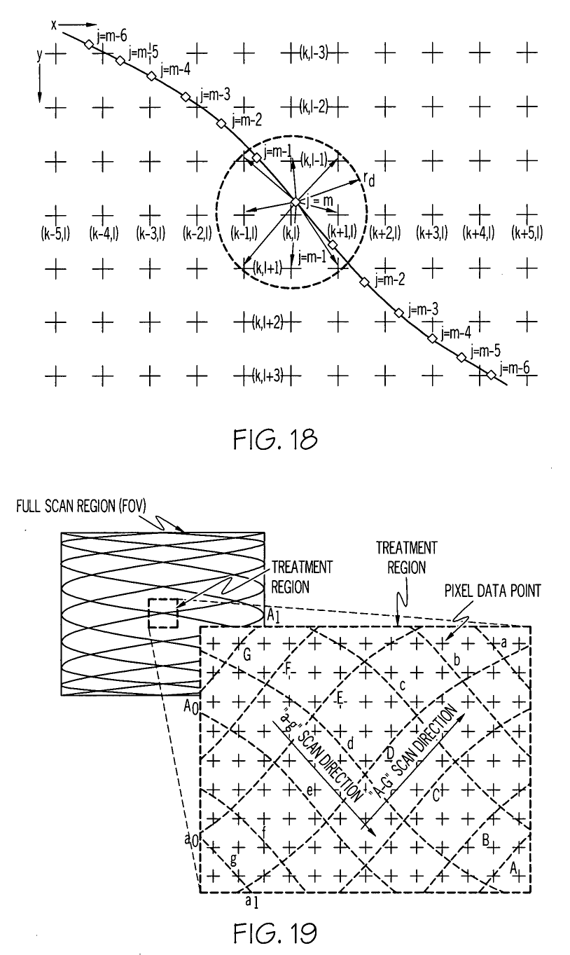

In FIG. 18, the beam trajectory (solid line) is shown overlaying pixel data (grey crosses). The index into the data samples is j and pixels have indices (k,l), corresponding to discrete values of conventional Cartesian coordinates (x,y): not matrix indices (row, column). The origin of the pixel data coordinates is in the upper left hand corner. Data from a particular data sample will be distributed into pixels falling into a region of radius r_{d} centered on the sample.

The solid line represents a portion of a specific trajectory of the dual resonant scanned beam through the scene. The diamonds indicate samples along that trajectory. The sample index (j) increases from the top left to bottom right in this depiction. The trajectory of the beam (with increasing sample index) can be in any direction through a subset of the scene. Note that the samples at the top left and bottom right are closer together than the samples in the center of the figure. This difference is shown to reinforce the implications of a constant data-sampling rate applied to resonant scanned beams. The particular sample index on the beam, m, will be utilized in subsequent discussions.

Conversion from Lissajous to Cartesian Data space can be represented as a matrix multiplication, followed by a data reordering

[SDV][T]=[PDV]

where the pixel data vector PDV is then reordered to yield the pixel data matrix PDM. If the number of samples in the SDV vector is N and the size of the Cartesian space is X by Y, the transformation matrix, T, is of dimension N by (X*Y).

The following process can be used to populate the T matrix. Through precise knowledge of the path of the scanned beam (that knowledge is assumed to be inherent in the scanner drive and positioning system) it is possible to identify the pixel data point closest to the sample, m, at t=mΔtₛ from the start of the frame. Denote that pixel with the indices (k,l). Next, construct a circle in Cartesian space of radius, r_{d}, over which the data from sample, m, is going to be distributed. For each pixel (k+s,l+t), where s and t are integers that describe points in Cartesian space located within the circle constructed above centered within the circle (a) compute the length (in Cartesian space), l, of the vector from the Cartesian space location of the SBI sample, m, to the center of the pixel space data pixel, (k+s,l+t) and (b) calculate a weighting value, w, that is proportional to the length, of the vector. Many functions can be used, however, it should be a function decreasing monotonically with distance, such as, for example,:

$$w = {e}^{- F \frac{s}{{r}_{d}}}$$

where:
w is the weighting factor,
s is the length of the vector from the SBI data point to the pixel of interest
F is a controllable constant that sets how fast the effects of the SBI data falls off as the value of 1 increases.
r_{d} is the radius of the circle over which the data from the SBI sample is being distributed.

Record the value of w into the transformation matrix T at the x,y location of the subject pixel. The location in the matrix will be at row m and column j*N+x. It should be recognized that this method creates a sparse matrix, T. To improve computational efficiency, one may optionally use various methods to create a banded matrix amenable to hardware acceleration or optimized software algorithms, which is described by Hammond S, Dunki-Jacobs R, Hardy R, Topka T. “Architecture and Operation of a Systolic Sparse Matrix Engine”, Proceedings of the Third SIAM Conference on Parallel Processing for Scientific Computing, 1987, (419-423), the details of which are hereby incorporated by reference as if fully set forth herein.

Display Coordinate to Scan Coordinate Mapping

One can convert from a particular data set, such as an image, in Cartesian space to a sample vector, m, by reordering the data into consistent form (that is, a vector of conformable size) and then solving the matrix equation:

[SDV]=[PDV]T⁻¹

where T is constructed as shown above. The above equation yields the multi-bit (analog) scan beam vector, SDV, which would result from a multi-bit (analog) Cartesian space matrix, PDM. Note that, in general, T is not square and the creation of the pseudoinverse matrix T⁻¹ can be computationally challenging, but can be accomplished as is known in the art. Distribution of multi-bit Cartesian space data to a multi-bit drive (continuously varying modulation) of the scan beam in Lissajous space does require construction of the inverse of the T matrix.

For simple ON/OFF control of the scan beam, however, the required mapping can be accomplished by simple inspection of the transformation matrix, T as follows. Each column,j, of the matrix, T, is associated with a specific Cartesian space location, (x,y), and contains the weighting function, w, for all of the samples in the vector SBV. Therefore, the m^{th} row in the column contains the weighting factor, w, for the m^{th}, sample in the vector SBV. As there might be multiple non-zero cells in the column, the closest sample to a particular location, (x,y), will be that row in the column with the largest value, w. By repeatedly performing the above inspection for each pixel (x,y) in the Cartesian space and placing the results at the appropriate location of a mapping matrix, M, that is of dimension Y by X; each cell of M contains the SBV sample number, m, closest to the Cartesian space location, (x,y).

FIG. 19 illustrates a simple case where a rectangular treatment region, a subset of the rectangular full FOV of the imaging system, is defined. In the expanded view of the treatment region, the pixel data locations (x,y), are denoted by the “+” symbols. The beam track is denoted by the long-dash lines (a-g,A-G). In this case, we wish to turn ON a treatment laser when the scan beam enters a treatment zone (e.g. at a₀ or A₀) and turn it OFF as the beam exits the treatment zone (e.g. at a₁ or A₁). It will be noted that the number of samples, and the amount of time, that the beam is ON changes from sweep to sweep.

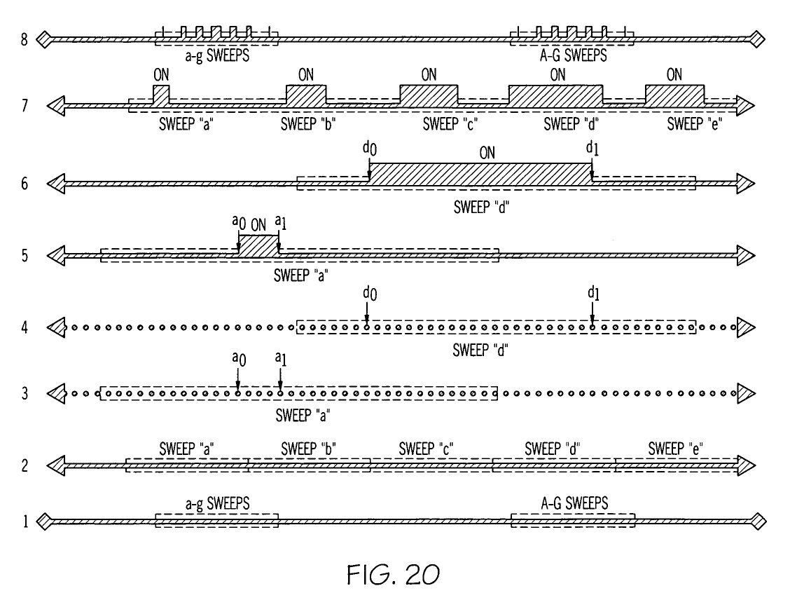

Referring now to FIG. 20, event timelines lead from a representation of the data stream or SBV (at the bottom of the figure, timeline 1) to a synchronized ON/OFF therapy or aiming source control stream (at the top of the figure, timeline 8) is depicted. The beam tracks of FIG. 19 are shown in a possible relationship to both the image data steam and treatment control stream. Note that it is not required that the temporal granularity (sampling period) of the data stream and the source control stream be identical. What is required is that the time from the start of the respective streams to the state changes in the source control stream and the target transition times in the data stream are reasonably similar.

In FIG. 20, a complete frame of the data stream of N samples is shown schematically in timeline 1. Within this timeline, the relative position and duration of the a-g and A-G sweeps is shown. Timeline 2 shows an expanded view of the a-e sweeps. Timeline 3 shows that the duration of a sweep (any of a-g and A-G) encompasses more time (samples) than the period during which the beam is in the treatment region (e.g. a₀ to a₁). Timeline 4 shows the same relationship for another beam d. Additionally, when inspecting beam ‘d’ path length vs. that of beam ‘a’ in FIG. 19, it is apparent that the time between the turn ON and turn OFF point is significantly different. That difference is shown schematically in timeline 4. Timelines 5 and 6 schematically represent the control signal that might turn ON and OFF a therapeutic or aiming source. Note that in timelines 3 and 4, the dotted nature of the lines shows that individual data samples are considered in this step. Note also that the spacing of samples is not shown for the analogous time scales of timelines 5 and 6. This emphasizes the fact that the granularity of the control timing does not have to match that available in the sampled data stream.

In light of the previous discussion of the mapping matrix M, it is clear that knowing the pixel locations, (x,y), at which the therapeutic or tracer beam enters and leaves the treatment zone can be computed and thereby, the times (from start of the frame) at which the control stream must turn ON and OFF (timeline 8 of FIG. 20).

There may be limitations on the minimum ON time for the source. Likewise, long ON times could cause system heating or other effects. These limitations might create situations where very short ON times are not honored and where long ON times might be broken into two or more patterns run on sequential frames.

A number of detailed embodiments have been described. Nevertheless, it will be understood that various modifications may be made. For example, in some embodiments, additional imaging beams and/or diagnostic beams are provided. For example, the source assembly 4 may be configured to provide radiation of a pre-selected wavelength, such as blue light (about 370 to about 460 nm), to excite tissue to autofluoresce or to excite an applied chemical to fluoresce. In other embodiments, an imaging beam may not be in the visible wavelength range, for example, a wavelength of about 1600 nm that may allow visualization of tissue structures through layers of other tissue or fluid or may enhance visualization of certain specific tissue types present in a field of blood or other tissue type. A complementary detector may be employed to detect the returned radiation and the controller is configured to display the signals in a chosen color or grayscale.

Scanner assembly 2 may also be used in a variety of skin surface treatments. For example, the scanner assembly 2 may be used for laser hair removal while reducing damage to surrounding skin. A medical device including the scanner assembly 2 may be used to produce an image of the skin surface, identifying a hair shaft, projecting the location and extent of the hair bulb, and the therapeutic laser can be automatically controlled to provide treatment to one or more of the hair shaft, hair follicle, hair bulb and dermal papilla. An acne reduction system can also be provided where the system including scanner assembly 2 is used to eliminate Propionibacterium acnes (P. acnes) bacteria while minimizing damage to surrounding skin. A medical device including scanner assembly 2 may be used to produce an image of the skin surface, identifying an acne site and the therapeutic laser can be automatically controlled to provide treatment. An acne reduction system can also be provided where the system including scanner assembly 2 is used to reduce local production of sebum while minimizing damage to surrounding skin. A medical device including the scanner assembly 2 may be used to produce an image of the skin surface, identifying an acne site, projecting the location of the sebaceous gland and the therapeutic laser can be automatically controlled to provide treatment. A skin rejuvenation system can also be provided including the scanner assembly 2 to precisely control laser-based thermal energy to small diameter, high aspect ratio treatment zones with substantial regions of untreated epidermal and dermal skin tissue in a manner that allows rapid, reliable skin rejuvenation, minimizing damage to surrounding skin tissue that can lead to prolonged post procedure recovery. This may be accomplished by verifying density (e.g., treatment zones per cm²) in an image obtained using a fixed focus scanner system of treatment zones applied to the skin. For portions of tissue that do not contain treatment zones of at least a user prescribed density, therapeutic laser pulses can be generated using the scanner assembly 2 to create additional treatment zones.

In some embodiments, the system may include tracking (e.g., using instrument motion sensors and tissue motion sensors) so that the targetable treatment region or point can move with moving tissue and/or a moving endoscope. In some embodiments, image recognition may be used for target tracking for example by looking for a distinctive feature in the image to act as a reference. In some embodiments, multiple, different control points or regions may be selected within the FOV, for example, to allow for treatment of multiple tissue areas as the reflector 27 moves. Accordingly, other embodiments are within the scope of the following claims.

What is claimed is:

1. A medical device comprising:
a radiation source assembly having at least two radiation sources, where one or more of the radiation sources is adapted to generate an imaging beam for use in visualization of a scene and one or more of the radiation sources is adapted to generate a therapeutic beam for treatment of a medical condition;
an optical fiber for directing radiation energy from the radiation source assembly toward a distal end of the medical device in the form of a beam;
a reflector that receives the beam from the optical fiber, the reflector configured to direct the beam onto a field-of-view; and
a receiving system including a detector arranged and configured to receive radiation from the field-of-view to generate a viewable image;
wherein the imaging beam and the therapeutic beam from the at least two radiation sources are directed to follow a common path to the reflector.

2. The medical device of claim 1, wherein the reflector oscillates at a natural resonant frequency in at least one axis.

3. The medical device of claim 1, wherein the therapeutic beam is generated based on user specification of at least one target region.

4. The medical device of claim 1 further comprising a controller that modulates sensitivity of the receiving system with delivery of the therapeutic beam to inhibit overload.

5. The medical device of claim 1, wherein the optical fiber is arranged and configured to receive an imaging beam and a therapeutic beam generated by the at least two radiation sources.

6. The medical device of claim 1 further comprising an image processor that generates a video image stream based on electrical signals generated by the detector that correspond to the radiation received by the detector from the field-of-view.

7. The medical device of claim 1 further comprising a display device for displaying a video image of the field-of-view to a user.

8. The medical device of claim 7, wherein the displayed video image is manipulated to illustrate a selected treatment region.

9. The medical device of claim 7 further comprising a motion sensing system including a motion sensor for use in detecting relative movement between the motion sensor and the field-of-view.

10. The medical device of claim 1, wherein the optical fiber is a single mode fiber.

11. The medical device of claim 1, wherein the radiation source assembly is configured to output an aiming beam to highlight an area of the field-of-view.

12. A method of providing medical treatment, the method comprising:
outputting an imaging beam using a first radiation source;
outputting a therapeutic beam using a second radiation source;
directing the imaging beam onto the field-of-view for generating a viewable image thereof using a reflector; and
directing the therapeutic beam onto at least a portion of the field-of view based on specification of a target region.

13. The method of claim 12 comprising outputting the therapeutic beam only when the reflector addresses the target region in the field-of-view.

14. The method of claim 12 further comprising directing the imaging beam and the therapeutic beam from the first radiation source and the second radiation source, respectively, to the reflector using a single mode fiber.

15. The method of claim 12 further comprising selecting the target region of the field-of-view based on user specification of a treatment zone.

16. The method of claim 15 further comprising defining the target region by mapping the user specified treatment zone to the field-of-view.

17. The method of claim 12 further comprising selecting the target region automatically by identifying a fluorescent material.

18. The method of claim 12 further comprising performing a photodynamic therapy, dermal treatment, thermal ablation or opto-thermal shock wave treatment by directing the therapeutic beam onto at least a portion of the field-of view.

19. The method of claim 12 further comprising directing an aiming beam onto the field-of-view, the radiation source configured to generate the aiming beam.

20. A medical device comprising:
a radiation source assembly configured to output an imaging beam and a therapeutic beam;
an optical fiber for directing at least one of the imaging beam and therapeutic beam toward a distal end of the medical device;
a reflector that receives at least one of the imaging beam and the therapeutic beam from the optical fiber, the reflector configured to direct the at least one of the imaging beam and the therapeutic beam onto a field-of-view;
a receiving system including a detector configured to receive radiation from the field-of-view to generate a viewable image; and
a user input device that allows for selection of a target region within the field of view.

21. The medical device of claim 20 further comprising a controller that controls operation of the radiation source such that a therapeutic beam is provided only within the target region.
